(19) United States
(12) Patent Application Publication
(10) Pub. No.: US 2002/0146401 A1
(43) Pub. Date: Oct. 10, 2002

(54) Generation and use of signal-plexes to develop specific cell types, tissues and/or organs

(76) Inventors: Eugene Bell, Boston, MA (US); Jianwu Dai, Boston, MA (US)

Correspondence Address: ELLEN LEONNIG, TEI BIOSCIENCES, INC., 7 ELKINS STREET, BOSTON, MA, 02127, US

(21) Appl. No.: 10/098,158

(22) Filed: Mar. 13, 2002

Related U.S. Application Data
(63) Continuation-in-part of application No. 09/901,765, filed on Jul. 9, 2001.
(60) Provisional application No. 60/251,125, filed on Dec. 4, 2000.

(51) Int. Cl. A61K 38/43; C12N 9/00; C12N 5/08

(52) U.S. Cl. 424/94.1; 435/183; 435/366

(57) ABSTRACT

Methods of forming and using signaling complexes (“Signalplexes”) expressed in specific tissues and organs during particular stages of development to predicably guide cell division, cell migration, differentiation of cells and/or formation of tissue or organ primordia in vitro.

Signal-plexes induce stem cells to express the same tissue and organ phenotypes as those from which the Signal-plexes were derived. Each member of the family of Signal-plexes induces differentiation of phenotypes only of a specific tissue or organ. Endocrine and exocrine pancreas, liver, lung, kidney, heart, cartilage, vascular, tendon, ligament and bone cells are induced in stem cells by Signal-plexes derived from each of the foregoing tissues as shown by morphology, immunostaining, ELISA, and RT-PCR analysis.

### CROSS-REFERENCE TO RELATED APPLICATIONS

This application is a continuation-in-part of U.S. Ser. No. 09/901,765, filed Jul. 9, 2001, the entire contents of which are incorporated herein by reference.

### BACKGROUND OF THE INVENTION

1. Field of the Invention

The use of human cells for the repair or replacement of defective parts of the body has been a key instrument of regenerative medicine and tissue engineering. The approaches have made use of fetal or post-natal cells propagated in vitro and delivered to a recipient in a scaffold that invites vascularization and, to some degree, recapitulates tissue or organ development (Bell, E. et al. Production of a tissue-like structure by contraction of collagen lattices by human fibroblasts of different proliferative potential in vitro. Proc. Natl. Acad. Sci. U.S.A. 76, 1274-8, 1979; Bell, E. et al. Living tissue formed in vitro and accepted as skin-equivalent tissue of full thickness, Science 211, 1052-4, 1982; Bell, E. et al. The reconstitution of living skin. The Journal of Investigative Dermatology 81, 2s-10s, 1983; Bell, E. et al. Reconstruction of a thyroid gland equivalent from cells and matrix materials. The Journal of Experimental Zoology 232, 277-85, 1984; Weinberg, C. B. et al. A blood vessel model constructed from collagen and cultured vascular cells. Science 231, 397-400, 1986).

The limited histiotypic potential of some of the foregoing specialized cells appears now to be only apparent; a skin graft made to the skin defines the signals as well as the phenotypic specificity expected of the graft. It is unexpected to discover that dermal cells of the skin, or some subset of them, possess a phenotypic potential much beyond that of topical fetal and post-natal skin (Toma, J. G. et al. Isolation of multipotent post-natal stem cells from the dermis of mammalian skin. Nat. Cell. Biology 3, 778-84, 2001).

Scientists are turning more and more toward stem cells as a resource for correcting deficits due to disease, injury or aging. While there has been progress in understanding how stem cells may be best used, and in probing the questions of the relative value of choosing embryonic, fetal or post-natal stem cells, the very basic requirement that stem cells need precise instructions to become a particular part of the body has remained unsatisfied. A major difficulty standing in the way of their most effective use is the need for signaling to direct the replication, morphogenesis and differentiation of embryonic, fetal or post-natal stem cells, that is cells of any age capable of responding to signals able to induce differentiation, leading to tissue or organ repair or regeneration.

Signaling in tissue development is a holistic process involving multiple signals. While complexes of tissue-specific signals are present and active in the course of tissue and organ development in the embryo, fetus and postnatal organism, the scope of these signaling complexes may change as tissues change in the course of development. During early development, groups of cells release specific signaling molecules that direct the differentiation of adjacent uncommitted cells. It is believed that each signaling complex is made up of many proteins. The relative proportions of these proteins; and the spectrum of different proteins present in each signaling complex, are responsible for cell divisions, cell migration, morphogenesis, differentiation, histiogenesis and organogenesis. It is also believed that the time span over which a signaling complex is expected to function is built into each signaling complex. In addition to signaling complexes usually secreted by cells and functioning outside of them, other factors, namely transcription factors activated by signaling complexes and functioning within the cells, also contribute to the developmental process.

2. Description of the Related Art

The ongoing discoveries of stem cell resources in the bodies of post-natal organisms and molecules capable of inducing stem cells to differentiate, greatly expand the promise of cell therapies and the possibilities for tissue and organ repair and regeneration. Cytokines and other signaling molecules have been shown to play a key part in initiating and accelerating tissue development, but the principal approach has been based mainly on the use of high doses of usually a single human recombinant product, at high cost. Another approach depends on the insertion of a cytokine gene, to upregulate output of a particular cytokine capable of improving tissue repair for example, VEGF to improve vasculogenisis. Although there have been some successes with the foregoing approaches, they are not fully physiologic.

Individual growth factors have been tested on mouse and human stem cells to identify the instructive molecules required to guide stem cell differentiation. Some studies show cytokine-induced differentiation of pluripotent embryonic stem cells grown as monolayers (Schuldiner, M. et al. Effects of eight growth factors on the differentiation of cells derived from human embryonic stem cells. Proc. Natl. Acad. Sci. U.S.A. 97, 11307-12, 2000). However, it has been concluded that none of the factors reported directs differentiation of a single cell type, nor has it been possible to predict a priori what effect one or more signaling agents will have on uncommitted stem cells.

Current work has depended heavily on the separation of cell types found in embryoid bodies (Stevens, L. Teratogenesis and spontaneous parthenogenesis in mice in The Developmental Biology of Reproduction, 93-106, Academic Press, New York, 1975) which develop after removal of leukemia inhibitory factor (LIF) from cultures of embryonic stem cells (Rohwedel, J. et al Muscle cell differentiation of embryonic stem cells reflects myogenesis in vivo: developmentally regulated expression of myogenic determination genes and functional expression of ionic currents. Developmental Biology 164, 87-101, 1994; Strubing, C. et al. Differentiation of pluripotent embryonic stem cells into the neuronal lineage in vitro gives rise to mature inhibitory and excitatory neurons. Mech. Dev. 53, 275-87, 1995). The myriad of specific factors involved in each step of cell, tissue and organ differentiation has yet to be identified. While it has been discovered by trial and error that certain facors can induce a particular ontogenetic change, the process of discovery is lengthy as well as risky, since the fine tuning due to the multiplicity of factors at work in vivo may not have been achieved.

### BRIEF DESCRIPTION OF THE INVENTION

Complexes of signaling molecules (“Signal-plexes) prepared from embryonic, fetal or post-natal animal tissue have been used to induce stem cells to express the same tissue and organ phenotypes as those from which the Signal-plexes were derived.

Signal-plexes are prepared by a method that involves harvesting, lysing, homogenizing and filtering animal tissue to remove solids to form extracts. The extracts are then fractionated. The term “extracts and fractions thereof” as used herein refers to biomolecules (e.g., proteins) that are substantially free of nucleic acids (e.g., DNA and RNA), cell membranes, nuclear membranes, nuclei, mitochondria and microorganisms. The biological activity of the extracts and fractions thereof are tested to select those which optimally induce differentiation of cells.

Stem cells form populations of differentiated cells upon being exposed to Signal-plexes. Endocrine and exocrine pancreas, liver, lung, kidney, heart, cartilage, vascular, tendon, ligament and bone formation are identified cytologically or histologically by characteristic morphology, immunostaining, ELISA, and RT-PCR analysis as shown infra. Cells induced to differentiate by Signal-plexes can be grafted (with the addition of a scaffold) or injected (with or without a scaffold) into a host to augment cell populations in tissue or organs requiring repair or regeneration. Signal-plexes can act as pharmaceutical agents and can be delivered by injection alone or with a carrier to tissues or organs harboring stem cells or progenitor cells to upregulate cell division and differentiation in the stem or progenitor cell population.

Signal-plexes have been used to identify a source of human stem cells (“Hu abba-1 cells”), as described in U.S. Ser. No. 09/901,786, the entire contents of which are incorporated herein by reference. Hu abba-1 cells are human fibroblasts extracted from the dermis of skin derived from fetuses between the ages of 8 and 24 weeks. Signal-plexes have also been used to discover stem cells in human post-natal skin cells.

### BRIEF DESCRIPTION OF THE DRAWINGS (PHOTOGRAPHS)

The patent or application file contains at least one drawing (photograph) executed in color. Copies of this patent or patent application publication with color drawing(s) (photograph(s)) will be provided by the U.S. Patent and Trademark Office upon request and payment of the necessary fee.

FIG 1a shows a subset of human stem cells derived from the dermis of fetal skin (“Hu abba-1 cells”). When cultured on plastic, Hu abba-1 cells resemble typical fibroblasts (phase contrast at magnification ×200).

FIG 1b shows uninduced control Hu abba-1 cells in collagen H-fiber scaffolds.

FIG 1c shows cartilage Signal-plex induced Hu abba-1 cells in fused collagen H-fiber scaffolds.

FIG 1d shows a histological section through fused scaffold (in FIG 1C) showing differentiated cartilage.

FIG. 2a shows an ELISA histogram of α1-AT secreted by Hu abba-1 cells induced by liver Signal-plex. B1, B3, and B5 are controls. B2 shows secretions by Signal-plex-induced cells in nodules; B4 and B6 of dissociated nodules.

FIG. 2b shows an ELISA histogram of serum albumin, a liver-specific marker. A1 and A3 are controls. A2 shows secretions by Signal-plex-induced cells in nodules; A4 is induced monolayered cells.

FIGS. 2c and 2d show Hu abba-1 and mES cells, immunostained for serum albumin.

FIGS. 2e and 2f show induced liver-specific α1-AT and serum albumin gene expression in Hu abba-1 cells, shown by RT-PCR. The second columns show bands induced by liver Signal-plex. The third and the fourth columns are negative and positive controls.

FIG. 3a shows aggregates of small cells arising from Hu abba-1 cells that have become epitheloid when Hu abba-1 cells are induced by pancreas Signal-plex.

FIGS. 3b and 3c show kidney Signal-plex-induced nodules stained yellow and pancreas nodules stained red. (FIG. 3c: magnification of ×100).

FIG. 3d shows ELISAs of insulin secretion by mES or Hu abba-1 cells in scaffolds. Sets 1 and 3 from left: uninduced; sets 2, 4, and 5: induced by pancreas Signal-plex. The blue bar measures insulin secreted in a three-week culture. The red bar is insulin output after addition of 8.0 mM glucose at three weeks; yellow at nine weeks.

FIG. 3e shows cells from a dissociated nodule, immunostained with anti-insulin antibody (magnification of ×630).

FIG. 4a shows uninduced Hu abba-1 cells (phase contrast at magnification ×100).

FIGS. 4b and 4c show kidney Signal-plex induced tubule-like structures from a three-dimensional nodule immunostained with rennin, a kidney-specific marker.

FIG. 4d shows the control.

FIG. 4e shows cells of a dissociated nodule immunostained with prekallikrein.

FIG. 4f shows nodules forming in a two-dimensional culture with surrounding epitheloid cells.

FIG. 4g shows extension of tubular processes from a nodule in a three-dimensional collagen primordium.

FIGS. 4h and 4i show nodules reconstituted from cells of a trypsin-dissociated nodule after 30 hours and 90 hours showing an increase of tubule-like structures.

FIG. 5a shows three-dimensional discs of collagen H-fiber seeded with Hu abba-1 cells and induced by bone Signal-plex. Only bone Signal-plex treated scaffolds or a BMP-2 positive control stained black (with Von Kossa stain) indicated formation of bone in vitro. Control without heart Signal-plex and control seeded with keratinocytes stained brown.

FIG. 5b shows nodules formed on a coverglass at 45 days after the confluent culture of Hu abba-1 cells was induced by heart Signal-plex.

FIG. 5c shows aggregates of mES cells induced to form cardiomyocytes under phase contrast. Aggregates of cells develop into beating structures that remained active for at least a month. This is an enlargement of one of the cell assemblies at magnification of ×200. 90% of the cells in the culture exhibited pulsitility.

FIG. 5d shows nodules of heart Signal-plex-induced Hu abba-1 cells immunostained positively for muscle actin.

FIG. 5e shows mES cell induced by heart Signal-plex and immunostained positively for muscle actin.

FIG. 6a shows a 20-day culture of Hu abba-1 cells in three-dimensional collagen H-fiber scaffolds, exposed to lung Signal-plex. FDA-stained living cells are organized into alveoli, while similar three-dimensional cultures of mES cells, three weeks after receiving lung Signal-plex, exhibit vascular networks consisting of vessels of various sizes (magnification is ×200).

FIG. 6b shows phase contrast view (magnification is ×200).

FIG. 6c shows living tissue stained with FDA (magnification is ×200).

FIG. 6d shows RT-PCR results: expression of the surfactant gene SP-A and of the gene for the vascular marker Flk-1 in induced Hu abba-1 cells.

FIG. 7a shows Hu abba-1 cells cultured for 12 days without vascular Signal-plex (the control).

FIG. 7b shows differentiated aggregate of Hu abba-cells exposed to vascular Signal-plex.

FIG. 7c shows RT-PCR analysis of two-dimensional and three-dimensional cultures in which differentiated aggregates developed. Two markers for arterial cells: a smooth muscle cell marker, smoothelin (SMTN), and the endothelial cell marker Flk-1 are shown, demonstrating induction of both phenotypes.

### DETAILED DESCRIPTION OF THE INVENTION

Stem cell populations have been discovered in fetal and post-natal skin cells through the use of Signal-plexes. The term “Signal-plexes” as used herein refers to signaling complexes that are expressed in specific tissues and organs during specific stages of development that can be used to predictably guide the differentiation of stem cells. Signal-plexes induce embryonic, fetal and post-natal stem cells to express the same tissue or organ phenotypes as those from which the Signal-plexes were derived. They comprise proteins or other biomolecules shown to promote cell division, morphogenesis and differentiation of specific tissues and or organs.

### Sources and Preparation of Signal-plexes

Signal-plexes can be derived from embryonic, fetal or post-natal animal tissue. They are developed by breaking, lysing homogenizing, and filtering tissue cells and subjecting the lysate to fractionation. Tissue is harvested, washed with buffer, and cut into small pieces. The buffer may be, for example, Tris buffer, HEPES buffer, or PBS, at a pH of 4.0-11.0, 4.5-10.5, 5.0-10.0, 5.5-9.5, 6.0-9.0, 6.5-8.5, 7.08.0, or preferably 7.4. The buffer preferably includes EDTA (at for example, 0-10 mM, 0.5-mM, or preferably 2 mM), and may also include protease inhibitors (for example, 1 mM PMSF and or 1 μM E-64). Preferably, the buffer is cold, e.g., about 4° C. The cut pieces of tissue are homogenized in buffer (preferably the same buffer that was used for washing), and extracts are obtained by collecting the supernatant after centrifugation at, for example, 17,000 g for 20 minutes, to remove particulate matter, including mitochondria and membranes.

A preferred example for preparing Signal-plex is as follows: a mammalian (preferably porcine) uterus is freshly removed and thawed at 4° C. for approximately 24 to 72 hours depending on the size of the uterus. The outside of the uterus is washed in 1% bleach solution and rinsed in sterile water. Each fetus in its intact amnion is removed from the uterus. Each fetus is recovered and briefly dipped into sterile PBS before its mass and length are measured.

Bone, brain, cord, cartilage, heart, kidney, liver, lung, muscle, pancreas, artery or aorta, is harvested and placed into sterile 50 ml centrifuge tubes or 250 ml beakers. The tissue or organ is washed three times in cold sterile PBS, and the volume is measured before the tubes are flash frozen for storage.

A homogenization buffer is added to the tissue (preferably, 2 ml of 50 mM Tris buffer is added per cc of tissue) at 4° C. If necessary, the tissue is divided into small pieces using an open blade generator for approximately 2 to 60 seconds. The tissue is homogenized near top speed for three bursts of 10 seconds with 10 mm or 20 mm micro ultrafine rotor/generator and chilled in their vessels for several seconds in cold methanol/dry ice. If necessary, 1 ml of homogenization buffer is added per 2 ml of remaining pellet; the pellet is re-homogenized and spun at 25,000 g (˜15,000 rpm) using a Beckman centrifuge (JA rotor) at 4° C. for 60 minutes. The foregoing step is repeated if necessary.

Signal-plex is filtered through a 0.451 μm syringe filter and then through 0.22 μm sterile filters to remove particulate matter and microorganisms (e.g., bacteria). Tubes are kept on ice in a sterile hood. 500 μl of each extract is retained for a protein assay. Between approximately 1.0 and 3.0 ml is aliquoted into sterile 1.7 ml eppendorf tubes. The tubes are flash frozen for storage.

### Cells Induced to Differentiate by Signal-plexes

Hu abba-1 cells; post-natal skin stem cells; and heart, kidney, liver, bone marrow, cartilage, ligament, tendon, muscle, adipose tissue, brain, lung, endocrine pancreas, neuronal stem or progenitor cells derived from human or experimental animals can be used to test the directive or inductive capacity of Signal-plexes. In another embodiment, pluripotent murine embryonic stem (mES) cells or cells derived from embryoid bodies (EBs) are used to test the inductive capacity of Signal-plexes using methods well-known in the art. Preferred sources of stem cells used to induce differentiation of cells upon exposure to Signal-plexes are: mouse embryonic stem (mES) cells derived from strain J-1, passage 13; human stem cells derived from the dermis of 8- to 10-week-old human fetal skin (e.g., Hu abba-1 cells); and post-natal skins stem cells derived from skin biopsies from foreskin or from skin of humans of any age.

Cell populations derived from any of the foregoing sources may be cultured on two-dimensional substrates with the addition of Signal-plexes. In a preferred embodiment, cells populations are cultured in three dimensions, for example in a collagen gel or fiber scaffold with a defined medium or low serum medium. Different types of biocompatible scaffolds having mechanical and other physical and chemical properties suitable for regeneration of different types of tissue are used. The period of time the cells are cultured ranges from approximately 1 day to about 14 months.

Collagen-based scaffolds resemble the native microenvironments which favor cell attachment, mobility and differentiation and tissue development; they provide a biocompatible substrate to which cells and cell products are able to bind. Preferably, collagen-based scaffolds as described in U.S. Ser. Nos. 5,800,537; 5,709,934; 5,893,888; 6,051,750; 09/871,518; 09/996,640, the entire contents of each are herein incorporated by reference, are used. For example, EBM, as described in U.S. Ser. Nos. 09/871,518 and 09/996,640, is a bioremodelable, biopolymer scaffold material prepared by subjecting animal tissue, particularly fetal or neonatal tissue, to chemical and mechanical processing. The process includes, but is not limited to, harvesting the tissue, optionally extracting growth and differentiation factors from the tissue, inactivating infective agents of the tissue, mechanically expressing undesirable components from the tissue, delipidizing the tissue, washing the tissue, optionally drying the tissue and optionally cross-linking the tissue not necessarily in the order described. The resulting product, EBM, is characterized by its microbial, fungal, viral and prion inactivated state. EBM is strong, drapable and does not undergo calcification. The scaffolds may also be of hydrated freeze-dried collagen or spun collagen fiber, or collagen gel using different types of collagen or other proteins or polymers (e.g., gelatin). If the collagen is crosslinked, the cross-linking procedure for scaffolds may be carried out by using a variety of chemical or physical cross linkers (e.g., lysyl oxidase, genipin treatment or UV irradiation).

The methods of adding stem cells to the scaffolds may vary. Stem cells may be added to freeze-dried scaffolds by hydrating the scaffolds with a cell suspension (e.g., at a concentration of about 100 to 1 million or more cells/ml of medium). Incorporation of cells into other types of scaffolds may be carried out by adding cells to a collagen solution, expected to form a gel at a temperature between 4° C. and 37° C. (preferably at 4° C. ). For example, Hu abba-1 cells, in a DMEM medium (10% FBS), or post-natal skin stem cells, are cultured in 12-well cell culture plates or on 12 mm coverglasses in 24-well plates. When 50-70% confluent, the serum is reduced to 0.5%.

The methods of adding Signal-plexes to the scaffolds may vary. Signal-plexes may be added when the freeze-dried scaffolds are manufactured or when tissue extracts and or fractions thereof are added to the culture directly. Signal-plexes may be added to a collagen solution or culture medium. The final concentration of Signal-plexes added to the cell cultures ranges between 1.0 μg/ml to 100 μg/ml of total protein. The medium is changed every 3-4 days with the addition of fresh Signal-plexes.

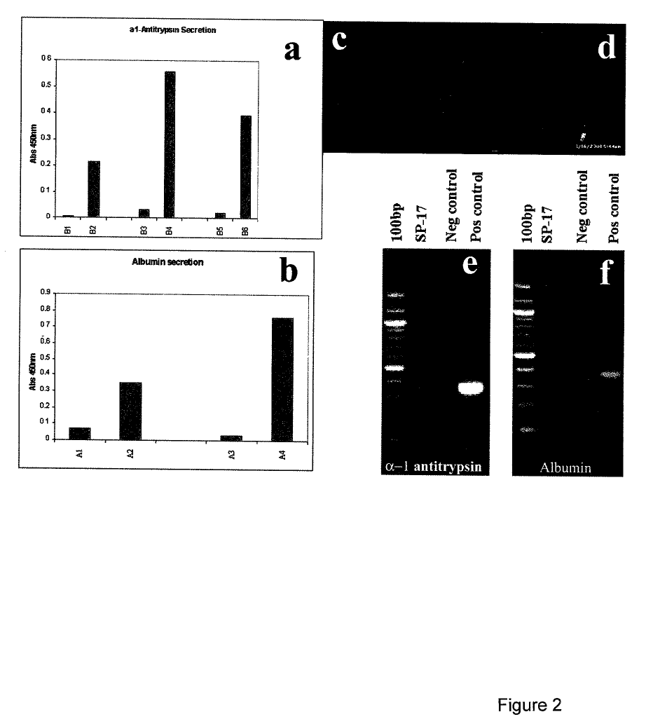

As stem cells arise, they form aggregates of small cells that undergo morphogenesis to become nodules (FIGS. 2a, 2b, 3b, 3c, 4f, 4h, 4i, 5b and 5d) from which differentiated cells are recovered. Cells derived from the nodules upon enzymatic dissociation of said nodules differentiate into new aggregates that form new nodules.

### Assaying Cells for Tissue-specific Properties

At the end of the culture period, the cells induced to differentiate by Signal-plexes are assayed for the cell or tissue types from which the Signal-plexes are derived. The cells may be assayed for the presence of one or more cell or tissue specific marker by, e.g., immunofluorescence or ELISA. In one embodiment, three dimensional scaffolds with cells may be processed for histological analysis. The term “tissue or organ primordia” as used herein refers to a combination of cells and scaffolds which lends itself to vascularization, remodeling and reconstitution of a functional replacement for a tissue or organ. In another embodiment, the cells may be assayed for expression of one or more tissue-specific mRNAs using Northern blotting or RT-PCR, methods which are well-known in the art. Products synthesized by gels induced by Signal-plexes can also be assayed by the ELISA procedure. Cells are assayed for tissue- or organ-specific products or are processed for histology after periods of between 2 days and several months.

Cells and tissues developing in separate cultures, recognizable as cartilage, bone, heart, lung, kidney, pancreas and liver can be induced to form in vitro and are identified are identified cytologically or histologically by characteristic morphologies or by functions, by ELISAs, by immuno- and other staining of products shown to be specific for the tissue, or by RT-PCR as described infra.

### Use of Signal-plexes for Tissue and/or Organ Repair or Regeneration

Signal-plexes can be used to make new body parts in vitro or to regenerate failing or malfunctioning body parts in vivo. For example, Signal-plexes can be used as pharmaceutical agents and can be delivered by injection alone or with a carrier. In a suitable carrier, such as a salve, ointment, or collagen scaffold, the pharmaceutical agent can be applied to the exterior or interior surface of the body. Signal-plexes can be used for wound healing. The term “wound” as used herein refers to cut, abrasion, burn, puncture, tear, break, fracture, ulcer or other tissue injury or loss of tissue integrity. Skin Signal-plex significantly reduces wound contraction in a rat model, compared with control grafts. It also promotes accelerated vascularization of scaffolds, and the accelerated population of scaffolds by neighboring cells which are attracted to migrate into it (e.g., dermal fibroblasts and keratinocytes). Signal-plex-enriched scaffolds are 30-100% faster than scaffolds not enriched with skin Signal-plex. The concentration of protein used in wound healing Signal-plex may be between 1.0 pg/ml and 20 mg/ml.

The use of Signal-plexes is not limited to repair or replacement of a specific tissue or organ. For example, specific fractions of lung or kidney extracts may also be used for repairing or regenerating other types of tissues, as well as the lung or kidney specifically, since both strongly promote angiogenesis. A heart Signal-plex may be used to promote regeneration of heart tissue before or after a heart attack since a population of progenitor cells that resides in the heart can be activated to divide by the Signal-plex. Appropriate Signal-plexes can be used for inducing regeneration of lung tissue in individuals with cystic fibrosis, or in both host and donor of portions of an organ, such as a lung or a liver, where increase of organ mass is desirable. The methods described herein can be applied not only to humans but also to animals of high economic or attachment value (e.g., race horses and pets).

### EXAMPLE 1

### Cartilage Tissue Formation In Vitro

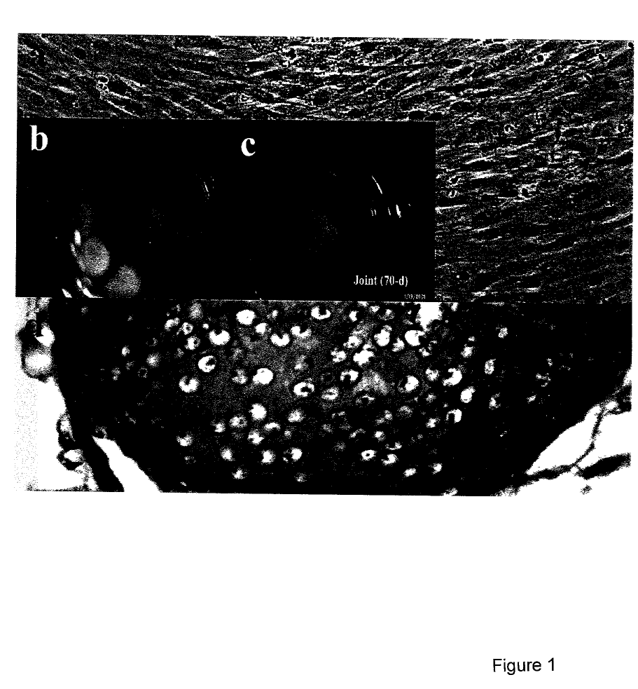

Cartilage forms from primordia when Hu abba-1 cells are seeded into three-dimensional scaffolds (preferably hydrated collagen fiber (H-fiber) as described in U.S. Ser. Nos. 09/519,247 and 10/037,149), freeze-dried and incubated with a cartilage Signal-plex (FIG. 1). Control cells are seeded into the scaffolds without the addition of Signal-plex. The cartilage scaffolds, now called primordia, exposed to cartilage Signal-plex, fuse over a period of two to three weeks to form larger masses (FIG. 1c). The control scaffolds remain apart. After 65 days of incubation, individual control scaffolds and the experimental fused primordia are processed for histology, sectioned, and stained with H&E. The section shown in FIG. 1d looks like typical articular cartilage. Histology of the control scaffolds show that the scaffolds are devoid of cells.

### EXAMPLE 2

### Expression of Liver Cell Markers In Vitro

### EXAMPLE 2A

### Expression of Liver Cell Markers in Hu abba-1 Cells

Upon exposure to the liver Signal-plex, Hu abba-1 cells form aggregates as well as nodules and express liver cell properties. ELISA demonstrates the biosynthesis of the liver-specific products, serum albumen and (α1-anti-trypsin (AT), (FIG. 2a and 2b). Neither variation of substrate nor growth of cells in two or three dimensions makes a significant difference in the capacity of cultures to synthesize either of the products. Immunofluoresence demonstrates liver serum albumin synthesis by both mES and Hu abba-1 cells when exposed to the liver Signal-plex (FIG. 2c and 2d). RT-PCR demonstrates the activation of the α1-AT gene and the serum albumin gene induced by the liver Signal-plex (FIG. 2e and 2f). The structure from which the tissue developed can be assembled with liver Signal-plex-induced cells dissociated from a nodule and seeded into a three-dimensional collagen fiber scaffold.

Total RNA can be extracted using a QIAGEN RNeasy RNA extraction kit. Reverse-transcription can be carried out with a Clontech Advantage RT-For PCR kit. Clontech's Titanium™ Taq PCR can be used with a two-step cycle at 68° as follows: 95 C. —1 min, 95 C. —30 sec (35 cycles), 68 C.—1 min, 68 C. —3 min. The primer sequences⁹ and PCR programs used for serum albumin and α1-AT are the same as reported (Meraw, S. J. et al, Treatment of peri-implant defects with combination growth factor cement. J. Periodontol. 71, 8-13, 1999).

For serum albumin: 5′-CCTTTGGCACAATGAAGTGGGTAACC; 3′-CAGCAGTCAGCCATTTCACCATAGG, expected product size=354 bp. For α1-AT: 5′-AGACCCTTTGAAGTCAAGGACACCG; 3′-CCATTGCTGAAGACCTTAGTGATGC, expected product size=360 bp. A Zeiss fluorescence microscope with a mercury light source and camera can be used to view the processed cells and tissues, as well as preparations of living tissues stained with FDA. ELISA is carried out by methods well-known in the art. Cultures are treated with the liver Signal-plex in 0.5 ml of Williams medium E, supplemented with 1% neutralized bovine calf serum, 4.5 g/L glucose, 7 ng/ml glucagon, 7.5 μg/ml hydrocortisone, 10 mM HEPES, 200 mM glutamine and IX Pen Strep. Cultures are incubated for 3-4 days, after which the media are collected and stored at −80° C.

### EXAMPLE 2B

### Expression of the Liver Phenotype (Stem Cells from Post-natal Skin Exposed to Liver Signal-plex)

Post-natal skin fibroblasts are exposed to liver Signal-plex. Cells are seeded on collagen coated-coverslips. Except for the substitution of the liver Signal-plex culture routines are the same as those for the post-natal skin cells treated with the pancreatic Signal-plex (described in Example 3B infra). At the two month time point, although no nodules form, since aggregates of the small cells develop, using the fibroblasts a feeder layer, that is growing on them, cells are tested for the expression of properties characteristic of the liver phenotype. Two monoclonal antibody markers are employed, one for serum albumin and the other for α1-anti trypsin. First the aggregates are trypsinized and the recovered cells in 10% FBS, DMEM are plated on collagen coated coverglasses as described supra. After two days, cells are fixed and immunostained with albumin and anti-trypsin monoclonal antibodies (1:200 dil). About 15% of the cells in the cultures stain positively with both antibodies. RT-PCR is performed which show bands for both serum albumin and α1-anti trypsin .

### EXAMPLE 3

### Expression of Pancreatic Cell Phenotypes In Vitro

### EXAMPLE 3A

### Expression of Pancreatic Cell Phenotypes in Hu abba-1 Cells

Upon exposure to pancreatic Signal-plex, Hu abba-1 cells express endocrine pancreatic cell phenotypes (e.g., glucagon producing and insulin producing) and exocrine pancreatic cell phenotypes. Functioning induced insulin-producing strains are kept in culture for nine weeks and longer in the presence of the pancreatic Signal-plexes.

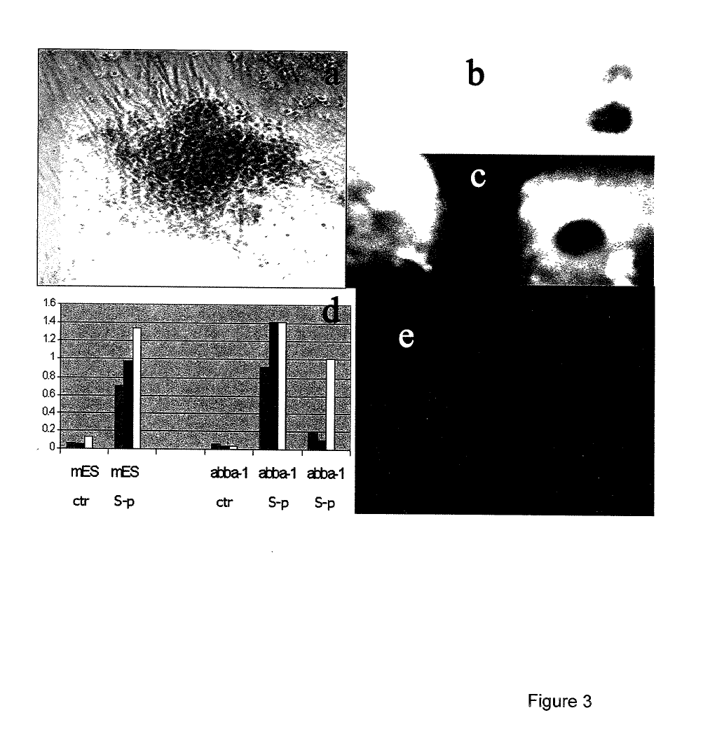

Hu abba-1 cells form aggregates when exposed to the pancreas Signal-plex in DMEM+0.5% FBS (FIG. 3a). In cultures that have formed nodules from aggregates, the nodules are recovered and assayed for the presence of insulin using dithizone (diphenylthiocarbozone) (Bonner-Weir, S. et al. In vitro cultivation of human islets from expanded ductal tissue. Proc. Natl. Acad. Sci. U.S.A. 97, 7999-8004, 2000), which stains insulin-containing cells red, and non-insulin containing cells yellow. FIGS. 3b and 3c show kidney Signal-plex-induced nodules stained yellow and pancreas nodules stained red. Zones in the nodules containing red-staining insulin-rich cells, stained yellow, suggest that the nodules consist of a mixed cell population. The nodule in FIG. 3c is a second-generation nodule arising from a dissociated first-generation nodule developed in a collagen gel scaffold. Individual cells from a trypsin-dissociated nodule stain red with the dithizone dye (not shown). ELISA tests of control cultures not enriched with Signal-plex and experimental cultures induced by the pancreatic Signal-plex show that both mES and Hu abba-1 cells are insulin secretors. Controls, which were not exposed to the Signal-plex, are negative. Ambient insulin secretion is compared with that upregulated by glucose at three and nine weeks (FIG. 3d). The cellular apparatus needed to sense 8.0 mM glucose is present, and both mouse and human cells respond by increasing insulin output (FIG. 3d). Cells from dissociated nodules immunostained with an anti-insulin antibody are shown in FIG. 3e.

Wells with cells expressing glucagon and chymotrypsin, respectively pancreatic endocrine and exocrine products (not shown), are also identified following exposure of Hu abba-1 cells to the pancreatic Signal-plex.

### EXAMPLE 3B

### Induction of Pancreatic Cell Phenotypes in Stem Cells Derived from Post-natal Human Skin

Post-natal dermal fibroblasts from the skin of a 25 year old donor at passage 4 (50,000 cells/ ml in DMEM, 10% FBS, Penicillin, Streptomycin and Fungizone, (PSF)) were plated on coverglasses coated with 50 μg/ml type I collagen (of rat-tail tendon origin) in 24 well non-adherent plates.

After 48 hours, standard medium is changed with reduced serum medium (DMEM, 0.5% FBS, P/S/F) containing 5-200 μg/ml of pancreatic Signal-plex (Signal-plex) prepared as described supra. Thereafter, cultures are fed with fresh medium containing Signal-plex twice a week. Control cultures are handled similiarly except that no Signal-plex is added.

After two months in culture, nodules of cells ranging in size between several hundred micrometers to 1.5 mm in diameter are observed in cultures fed Signal-plex. As many as 50 or more nodules were observed on each cover glass. Nodule formation is preceded by the morphogenesis of dense aggregates of small spherical or polyhedral cells that were growing on the dermal fibroblasts that appeared to be serving as a feeder layer. Cultures are not passaged during the period of development of aggregates and nodules. Nodules developing in cultures of post-natal fibroblasts, exposed to kidney Signal-plex under the foregoing method, serve as negative controls for analysis of markers for the pancreatic phenotype. On staining both kidney nodules and pancreatic nodules with dithizone, a specific stain for insulin, pancreatic nodules stain bright red as expected if the constituent cells or some portion of them are synthesizing insulin. Kidney nodules stained yellow give no evidence of insulin biosynthesis.

In preparation for experiments designed to assess the expression of the gene coding for insulin by means of RT-PCR, cells as well as aggregates growing on coverglasses for a period of several weeks are removed from the coverglasses with 1 mg/ml collagenase type I (Worthington biochemical Corp. NJ) in buffer containing 50 mM Tris, 5 mM CaCl₂, pH 7.5. Cells from the coverglasses as well as cells dissociated from nodules present on the coverglasses are washed with 0.5% FBS, DMEM, P/S/F and the cell pellets are resuspended in the same medium. Collagen H-fiber scaffolds are soaked overnight in 5% FBS, DMEM, P/S/F and dried on sterilized filter paper. The scaffolds measuring 4 mm² by 2 mm high are immersed in a suspension of the cells removed from the cover glasses and delivered to a 15.0 ml centrifuge tube in the foregoing medium except that the serum concentration is reduced to 0.5%. Scaffolds are gently shaken by inverting capped tubes 10 times in the course of several minutes, so that cells are well seeded into the scaffolds. The scaffolds containing cells are incubated in 24 well non-adherent plates that are transferred to a 37° C., 7% CO₂ incubator. The remaining cells in the centrifuge tubes are equally distributed among the wells containing scaffolds. After three days, cells that are not attached to scaffolds form aggregates on the bottom of the wells, and are transferred for additional culturing as described infra. Medium in which the scaffolds are incubated is changed after three days; pancreatic Signal-plex is added and a schedule of bi-weekly medium changes containing Signal-plexes is initiated.

After three weeks (that is, three weeks plus two months), the cells residing in the scaffolds are used as a source of mRNA, and RT-PCR is carried out on the product. Results are negative for control cultures not treated with Signal-plex and for m-RNA derived from similar cultures in which the kidney phenotype is induced. RT-PCR for expression of the insulin gene in cultures exposed as described to the pancreatic Signal-plex show a strong band for the insulin gene coincident with the positive control band given by cells from the pancreas itself.

### EXAMPLE 3C

### Segregation of Pancreatic Endocrine and Exocrine Cells (Stem Cells from Post-natal Skin Exposed to Pancreatic Signal-plex)

The aggregates of cells removed from wells containing scaffolds as described above are delivered to wells of 24 well tissue culture plates on which a thin layer of collagen gel (0.5 mg/ml in 0.5% FBS, DMEM; 500 μ/well) is prepared. For experimental wells, the Signal-plex solution is incorporated into the collagen gel. Control cultures, in 0.5% serum plus the usual medium, established at the same time that the original cover glasses are plated, provide cells plated onto collagen gels that do not contain Signal-plex. They serve as negative controls. After seven days in culture, aggregates of cells form between the gel and the bottom of the well. The aggregates of triangular cells include groups organized into acinar-like structures. The cells are rich in granules of a size and distribution resembling zymogen granules. Cells derived from control cultures in which the coverslip was treated with only 0.5% FBS, DMEM, P/S/F but not Signal-plex do not exhibit exocrine cell properties. The cells from under the gels are trypsinized and regrown in 0.5% FBS, in standard medium on collagen coated coverslips for two hours and fixed with 3.7% formaldehyde. They are immunostained with an amylase monoclonal antibody (1:200 dil) using methods well-known in the art. Cells in the culture stain positively for the antigen; controls do not stain. RT-PCR for expression of the amylase gene is positive.

### EXAMPLE 3D

### Insulin Staining of Cells and Aggregates Which Remained on the Surface of the Foregoing Collagen Gels

Cells remaining on the surface of the gel form aggregates and nodules within two weeks. Upon staining with dithizone, all nodules and a small percentage of cells not associated with nodules stain positively. None of the cells or aggregates recovered from under the gel stain positively for dithiazone. The method for separating the endocrine and exocrine cells, particularly the nodules which develop from cells plated on the surface of the collagen gel, can be used for scaling up the production of insulin-producing cells.

### EXAMPLE 4

### Expression of Kidney Cell Phenotypes In Vitro

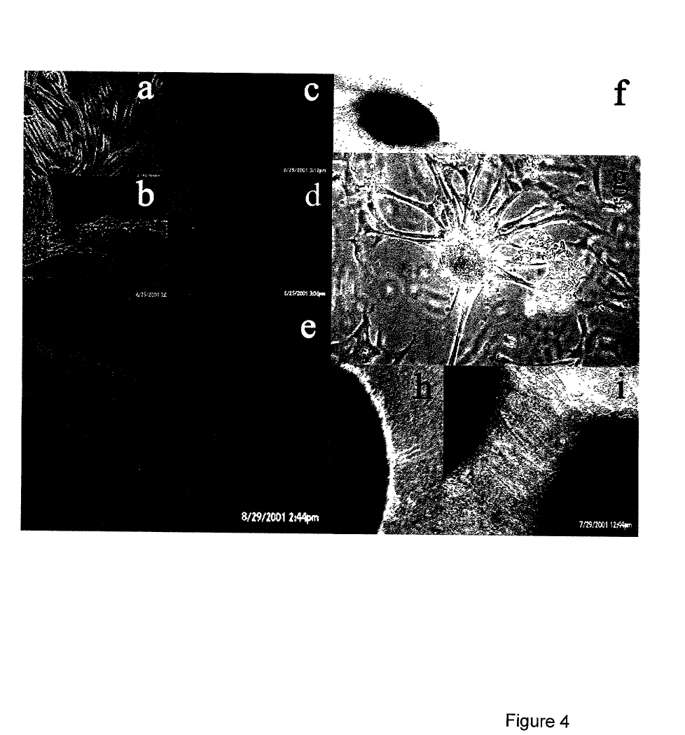

After mES or Hu abba-1 cells are exposed to the kidney Signal-plex, the foci of cells which form are epitheloid and express prekallikrein, a serine protease, shown by immunostaining (FIG. 4d, magnification x100; control is 4c), found in some other organs as well as the kidney and present in serum. FIG. 4a is an uninduced Hu abba-1 control, while FIG. 4b is a phase contrast view of the induced nodule from which tubular processes are extended. Moreover, the induced cells express renin (FIG. 4e, magnification ×630), a kidney-specific enzyme that is secreted into the blood, where it cleaves angiotensinogen. Renin is synthesized by cells in the juxtaglomerular region of the nephron, where the ascending straight portion of the distal tubule returns to the renal corpuscle.

The aggregates of cells form into three-dimensional nodules of various sizes, ranging between 0.1 mm and 1 mm (FIGS. 4b and 4f-i). The nodules are dissociated with trypsin, replated, and observed to recapitulate the process of aggregate formation and expression of the kidney phenotype. If the aggregates are cultured in a three dimensional collagen scaffold, they extend processes that are microvessel-like (FIGS. 4g and 4i, magnification ×100) and are referred to herein as “tubule-like processes”. In the normal development of the kidney, the entire vasculature of the organ arises from the metanephric primordium (Abrahamson, D. R. et al. Origins and formation of microvasculature in the developing kidney. Kidney Int. 54, S7-11 (1998). Fragments of mouse embryonic kidney explants cultured under serum-free conditions in vitro, in the presence of the phorbol ester TPA, extend arrays of what have been interpreted as microvessels (Antes, L. M. et al. A serum-free in vitro model of renal microvessel development. A. J. P. Renal Physiology 274, F1150-60 (1998) resemble the tubule-like processes. Aggregates of kidney Signal-plex-induced Hu abba-1 cells in a collagen gel in wells of 24-well plates extend processes, which lengthen over a period of 90 hours. Length of the structures increases between 30 and 90 hours of incubation (FIGS. 4h and 4i).

The capacity of nodules to extend tubular structures persists for periods of two to three weeks. None of the aggregates or nodules that arise from the use of other Signal-plexes induce formation of similar structures.

### EXAMPLE 5

### In Vitro Bone Induction

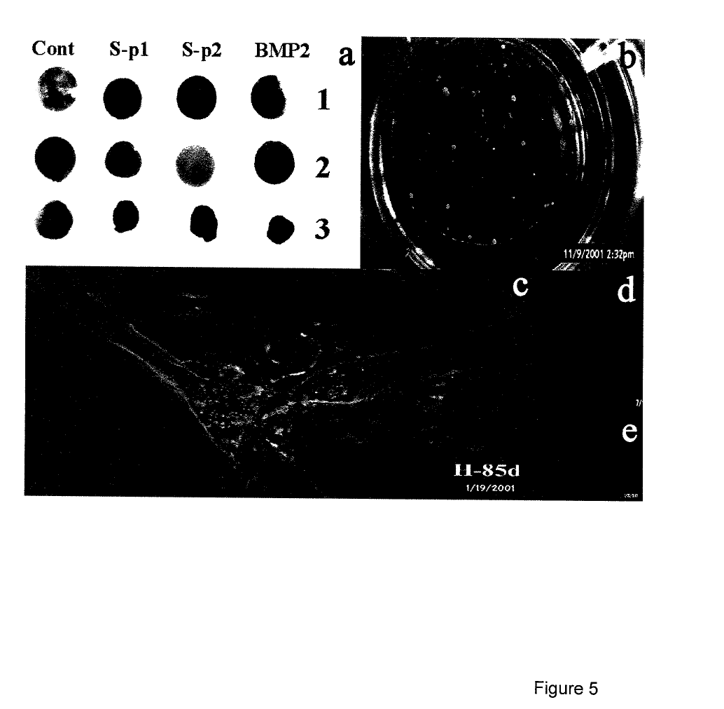

Bone primordia arising from mES or Hu abba-1 cells (FIG. 5a, top and bottom rows respectively) are seeded into collagen fiber scaffolds, cast in the form of discs 8 mm in diameter, and incubated with two variations of bone Signal-plex or BMP-2. By eight weeks, they positively stain black by von Kossa bonespecific staining. Similar results are obtained using mES or Hu abba-I cells (FIG. 5a). The control collagen scaffold discs receiving bone Signal-plex or BMP-2 and seeded with keratinocytes (FIG. 5a, row 2) stain mainly red, while mES and Hu abba-1 controls not receiving bone Signal-plex (FIG. 5a, left column) stain brown, not black as expected. Neither they nor the control discs containing mES or Hu abba-1 cells without Signal-plex or BMP-2 harden to the touch, as do the experimental discs.

### EXAMPLE 6

### Expression of Cardiomyocyte Markers In Vitro by Heart Signal-plex

Heart Signal-plex induces both mES and Hu abba-1 cells to express heart muscle actin as seen by immunostaining with antiheart muscle actin. (FIG. 5d is an Hu abba-1 aggregate, magnification ×100 and FIG. 5e is an mES cell, magnification ×630.) The mES cells form aggregates (FIG. 5b and 5c), which begin to beat in vitro after about 1-2 months, suggesting highly efficient differentiation to cardiomyocytes. 90% of cells in the mES cultures exhibit pulsitility.

### EXAMPLE 7

### Effects of Lung Signal-plex on Hu abba-1 Cells

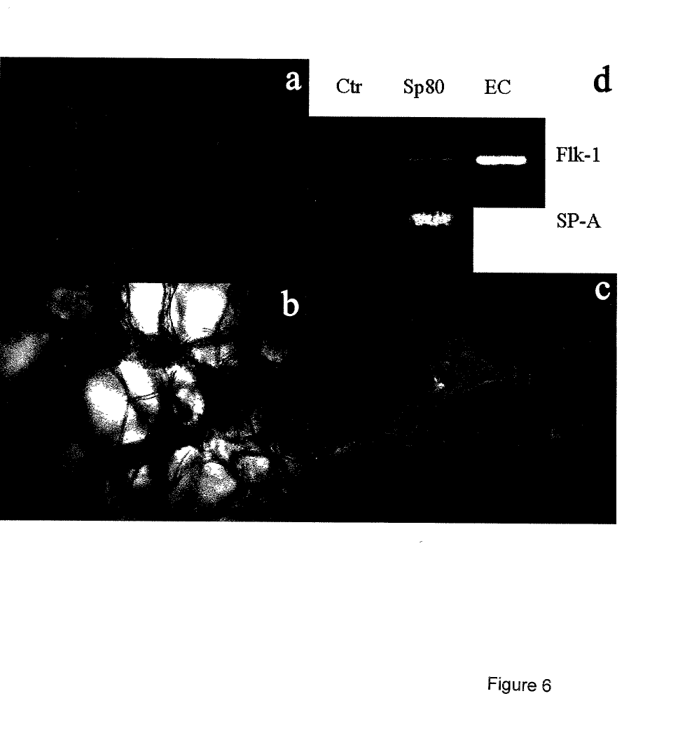

Hu abba-1 cells are seeded into a three-dimensional collagen fiber scaffold and exposed to the lung Signal-plex. Alveoli form in an organotypic array, shown by fluorescein diacetate (FDA) stained in FIG. 6a. The space gaps are respected and preserved by the cells that maintain the alveolar structure. Uninduced control cells seeded into similar scaffolds grow in the scaffold matrix and by 20 days have overgrown it randomly (not shown). When plated onto a plastic cell culture surface cells organize themselves into a planar alveolar network. In a three-dimensional collagen fiber primordium, mES cells exposed to the lung Signal-plex develop a system of vessels of multiple sizes by two weeks, as shown by FDA staining in the living state (FIG. 6c) and in phase contrast (FIG. 6b). The vasculogenesis induced by the lung Signal-plex is considered to be a “conspicuous feature” of the forming alveolar wall (Bloom and Fawcett, Histology, Chapman & Hall, New York, ed. 12, 1994).

RT-PCR is carried out using Qiagen Omniscript Reverse Transcriptase and HotStar Taq DNA Polymerase was used for the PCR protocol. Primers developed for Flk-1 are 5′-GCTCAGCATACAAAAAGACATACTT; 3′-ACTCAGAACCACATCATAAATCCTA, expected product size=589 bp. Primers for the surfactant SP-A are 5′-AGAAATGCCATGTCCTCCTG; 3′-TTCCACTGCCCATCTGTGTA, expected product size=510 bp. Primers for human smoothelin are 5′-CAGGCCGAGAAGAAGAAAGA and 3′-CACACAGTCCACCAGCATCT and the predicted product is 399 bp. Initial activation step is 15 min at 95° C. followed by three-step cycling: denaturation 1 min at 95° C.; extension is 1 min at 72° C. for 30 cycles; final extension is 10 min at 72° C.

RT-PCR analysis for the pulmonary surfactant protein A (SP-A) (FIG. 6d), secreted by the great alveolar cells as a monolayer coating the alveolar wall, demonstrates the induced activity of the gene which codes for it. The vascular gene Flk-1 is also seen to be expressed (FIG. 6d).

### EXAMPLE 8

### Vascular Signal-plex Induces Two Arterial Phenotypes

Embryonic stem cells can be induced to express both endothelial cell markers and smooth cell markers (Carmeliet, P. One cell, two fates. Nature 408, 43-44, 2000). Upon exposure to the vascular Signal-plex, Hu abba-1 cells can be induced to do the same.

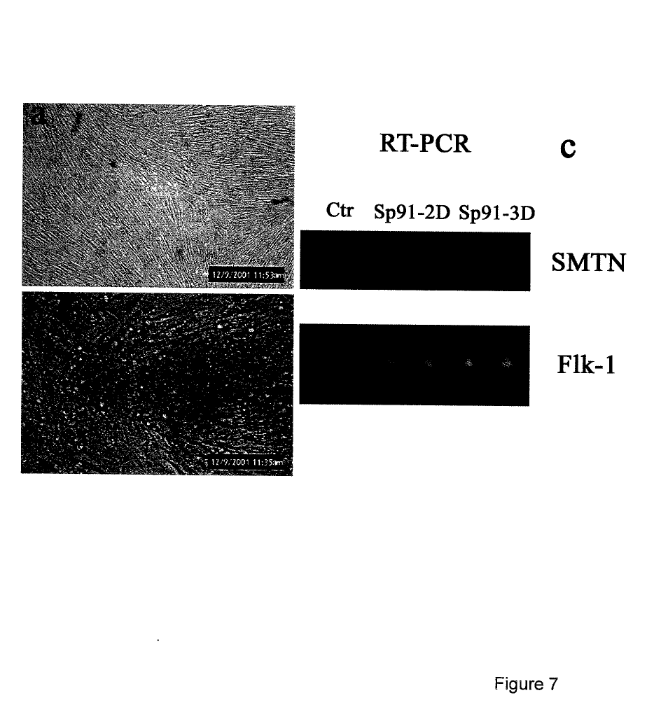

When Hu abba-1 cells are cultured in two-dimensions and in three-dimensional H-fiber collagen scaffolds, and treated with the vascular Signal-plex, aggregates of differentiated cells occur after 12 days (FIG. 7b; control is 7a). When RT-PCR is carried out using the Flk-1 gene as a marker for endothelial cells and smoothelin (SMTN) as the marker for smooth muscle cells (Van der Loop, F. T. et al. Differentiation of smooth muscle cells in human blood vessels as defined by smoothelin, a novel marker for the contractile phenotype. Arteriosclerosis, Thrombosis, and Vascular Biology 17, 665-671, 1997), cells express both markers (control is FIG. 7c).

Table 1 provides the sizes and numbers of nodules of specific phenotypes in cultures induced by different Signal-plexes. All cells of the dissociated nodules exhibit a marker that identifies the phenotype expected to be induced by the Signal-plex used. When the cultures are covered with a 0.5% collagen gel, fusion of nodules arising from aggregates occurs.

TABLE 1
Number of nodules on coverslips (n = 2-4) by size and Signal-plex concentration;
number of aggregates as a function of Signal-plex concentration.

| Signal-plex | Signal-plex (μg total protein) | ≦0.5 mm | ≧0.5 mm | Aggregates |
|---|---|---|---|---|
| Liver | 75 | 0 | 15 | 25 |
| Liver | 150 | 5 | 21 | 41 |
| Lung | 50 | 11.5 | 23 | 39 |
| Heart | 50 | 19.5 | 56.5 | 65 |
| Pancreas | 100 | 5.5 | 20 | 11 |
| Kidney | 75 | 2.5 | 6 | 18 |
| Kidney | 150 | 10 | 6 | 12.5 |

### Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, issued patents, and published patent applications cited throughout this application are hereby incorporated by reference. The appropriate components, processes, and methods of those patents, applications and other documents may be selected for the present invention and embodiments thereof.

What is claimed is:

1. A method for preparing Signal-plexes comprising:
a) harvesting animal tissue;
b) lysing, homogenizing and filtering said tissue to produce extracts;
c) fractionating said tissue to produce extracts and fractions thereof; and,
d) testing the biological activity of said extracts and fractions thereof to select those which optimally induce differentiation.

2. The method of claim 1, wherein said tissue is selected from the group consisting of embryonic, fetal and post-natal tissue.

3. The method of claim 1, wherein said solids comprise cell membranes, nuclear membranes, nuclei and mitochondria.

4. A Signal-plex prepared by the method of claim 1.

5. A Signal-plex comprising biomolecules that are capable of inducing cells derived from animal tissue to differentiate and form tissue and organ primordia in vitro and are substantially free of nucleic acids, cell membranes, nuclear membranes, nuclei, mitochondria and microorganisms.

6. A method for differentiating cells to form tissue and organ primordia in vitro comprising:
a) providing cells derived from animal tissue;
b) delivering said cells to a substrate;
c) cultivating said cells in a culture medium with serum;
d) providing Signal-plex(es); and,
e) exposing said cells to said Signal-plex(es) for a period of 1 day to 14 months.

7. The method of claim 6, wherein said tissue is selected from the group consisting of embryonic, fetal and post-natal tissue.

8. The method of claim 6, wherein a subpopulation of cells in said tissue are progenitor cells and stem cells.

9. The method of claim 8, wherein said cells are fetal dermal fibroblasts harboring a subpopulation of said stem cells called Hu abba-cells.

10. The method of claim 8, wherein said cells are post-natal dermal fibroblasts from a person of any age harboring a subpopulation of said stem cells not named.

11. The method of claim 8, wherein said progenitor and stem cells in embryonic, fetal and post-natal tissues are induced to form when exposed to said Signal-plex(es).

12. The method of claim 6, wherein said substrate is selected from the group consisting of a cover glass, tissue culture plate, collagen fiber scaffold and collagen gel scaffold.

13. The method of claim 6, wherein said delivering comprises adding said cells to a scaffold.

14. The method of claim 11, wherein said scaffold is three-dimensional.

15. The method of claim 11, wherein said scaffold comprises at least one type of collagen.

16. The method of claim 13, wherein said scaffold is selected from the group consisting of a hydrated collagen fiber, collagen gel, collagen foam, freeze dried collagen and EBM.

17. The method of claim 6, wherein said cultivating does not comprise subculturing.

18. The method of claim 6, wherein said cultivating comprises renewing said Signal-plex(es) and said medium every 3 to 4 days.

19. The method of claim 6, wherein said cells exposed to Signal-plex(es) form aggregates of small cells that undergo morphogenesis to become nodules; and, cells derived from said nodules upon enzymatic dissociation of said nodules differentiate into new aggregates that form new nodules.

20. The method of claim 6, wherein said exposing comprises exposing said cells to one of said Signal-plex(es) at a time.

21. A cell(s) differentiated by the method of claim 6.

22. A cell(s) of claim 21, wherein said cell(s) is selected from the group consisting of endocrine and exocrine pancreas, liver, lung, kidney, heart, cartilage, vascular, tendon, ligament and bone cells.

23. A tissue primordium formed from a cell(s) of claim 21.

24. An organ primordium formed from a cell(s) of claim 21.

25. A cell(s) of claim 21, wherein said cell(s) is selected from the group consisting of endocrine and exocrine pancreas, liver, lung, kidney, heart, cartilage, vascular, tendon, ligament and bone cells.

26. A method for using Signal-plexes to treat a patient comprising delivering said cell(s) of claim 21 and tissue and organ primordium formed by said cell(s) to a patient, wherein said patient is selected from the group consisting of a human, an animal of high economic value, and an animal of high attachment value.

27. The method of claim 26, wherein said delivering comprises grafting said cell(s) and tissue and organ primordium formed by said cell(s) into said patient.

28. The method of claim 26, wherein said delivering comprises injecting said cell(s) and tissue and organ primordium formed by said cell(s) into said patient.

29. A method of using cells dissociated from aggregates of small cells for promoting cell divisions and scaled-up propagation of differentiated cells in vitro comprising:.
a) providing cells of claim 19;
b) cutting out aggregates of small cells by using a device selected from the group consisting of a pair of fine scissors and cloning ring;
c) dissociating said aggregates of small cells enzymatically into single cells to create a suspension of cells;
d) diluting said suspension of cells;
e) plating said cells at low density;
f) culturing and passaging said cells in the presence of leukemia inhibitory factor (LIF) in a bioreactor to increase the numbers of said cells; and,
g) exposing said cells to Signal-plex(es) after the removal of said LIF to form fully differentiated cells.

30. A differentiated cell(s) formed by the method of claim 29.

31. A tissue primordium formed by a differentiated cell(s) of claim 30.

32. An organ primordium formed by a differentiated cell(s) of claim 30.

33. A method for using differentiated cells for treating a patient by delivering a cell(s) of claim 36, and tissue and organ primordium formed by said cell(s) to a patient, wherein said patient is selected from the group consisting of a human, an animal of high economic value, and an animal of high attachment value.

34. The method of claim 33, wherein said delivering comprises grafting said cell(s) and tissue and organ primordium into said patient.

35. The method of claim 33, wherein said delivering comprises injecting said cell(s) and tissue and primordium into said patient.

36. A method for identifying stem cells in animal tissue comprising:
a) providing a cell(s) of claim 21;
b) assaying said cells for tissue-specific properties.

37. The method of claim 36, wherein said assaying comprises performing a test selected from the group consisting of morphology, immunostaining, ELISA and RT-PCR.

38. A stem cell identified by the method of claim 36.
